(19) United States
(12) Patent Application Publication
(10) Pub. No.: US 2025/0339270 A1
(43) Pub. Date: Nov. 6, 2025

(54) TRANSCATHETER DEVICES AND METHODS

(71) Applicant: Medtronic, Inc., Minneapolis, MN (US)

(72) Inventors: Sydney M. Gustafson, San Francisco, CA (US); Christina E. Barbaro, San Francisco, CA (US); Lauren A. Safai, Los Altos, CA (US); Matthew C. Ziebol, Lino Lakes, MN (US); Suryansh Kumar, Rohnert Park, CA (US)

(21) Appl. No.: 19/194,111

(22) Filed: Apr. 30, 2025

Related U.S. Application Data
(60) Provisional application No. 63/643,346, filed on May 6, 2024.

(51) Int. Cl. A61F 2/24

(52) U.S. Cl. CPC A61F 2/2436; A61F 2/2418

(57) ABSTRACT

A transcatheter device for delivering a stented prosthesis includes a sheath shaft comprising a proximal end and a distal end. The transcatheter delivery device can further include a capsule attached at a distal end of the sheath shaft. The capsule comprises an outer circumferential surface and at least one protrusion extending radially outwardly from the circumferential surface. The transcatheter delivery device further includes an inner shaft with a proximal and distal end as well as a distal tip attached to a distal end of the inner shaft. The distal tip is configured to close a distal end of the capsule while the stented prosthesis is mounted on the inner shaft within the interior of the capsule. Methods are also provided for deploying a stented prosthesis wherein leaflets are displaced and then the displaced leaflets are pinned in position by the deployed stented prosthesis.

### CROSS-REFERENCE TO RELATED APPLICATIONS

This application claims the benefit of U.S. Provisional Patent Application Ser. No. 63/643,346, filed May 6, 2024, the entire content of which is incorporated herein by reference.

### FIELD

The present disclosure relates generally to transcatheter devices and methods, and more particularly to transcatheter devices and methods of deploying a stented prosthesis within a heart valve.

### BACKGROUND

A human heart includes four heart valves that determine the pathway of blood flow through the heart: the mitral valve, the tricuspid valve, the aortic valve, and the pulmonary valve. The mitral and tricuspid valves are atrio-ventricular valves, which connect the atria to the ventricles, while the aortic and pulmonary valves are semilunar valves located between the ventricles and their corresponding artery and regulate the flow of blood leaving the heart. Each of the valves are made from thin, strong flaps of tissue called leaflets. Ideally, native leaflets of a heart valve move apart from each other when the valve is in an open position and meet or “coapt” when the valve is in a closed position.

Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Diseased or otherwise deficient heart valves can be repaired or replaced using a variety of different types of heart valve surgeries. One conventional technique involves an open-heart surgical approach that is conducted under general anesthesia, during which the heart is stopped, and blood flow is controlled by a heart-lung bypass machine.

More recently, minimally invasive approaches have been developed to facilitate catheter-based implantation of a prosthetic heart valve or prosthesis on the beating heart, intending to obviate the need for the use of classical sternotomy and cardiopulmonary bypass. In general terms, an expandable prosthetic valve is compressed about or within a catheter, inserted inside a body lumen of the patient, such as the femoral artery, and delivered to a desired location in the heart.

However, the minimally invasive approaches are sometimes less effective for patients who have uncommonly large valve leaflets. When the prosthetic heart valve is deployed, these leaflets can cause coronary instruction where the leaflets block the replacement valve when it opens and prevents the flow of blood to the coronary arteries. Further, the prosthetic heart valves over time may become damaged or diseased from stenosis and may need replaced. Catheter-based approaches have also been used to repair or replace the damaged prosthetic heart valves, but similarly the leaflets from the previously implanted prosthetic heart valve can cause coronary obstruction.

In light of the above, a need exists for a transcatheter device that can manage the leaflets of a heart valve during catheter-based implantations of prosthetic heart valves.

### SUMMARY

The following presents a simplified summary of the disclosure to provide a basic understanding of some aspects described in the detailed description.

In aspects, transcatheter devices comprise an outer shaft and a capsule attached to a distal end of the outer shaft. The capsules comprise an outer circumferential surface and at least one protrusion extending radially outwardly from the outer circumferential surface. The transcatheter devices further comprise an inner shaft extending within the outer shaft, and a distal tip attached to a distal end of the inner shaft, wherein the distal tip is configured to close a distal end of the capsule.

In further aspects, transcatheter devices comprise an outer shaft and a capsule attached to a distal end of the outer shaft. The transcatheter devices further comprise an inner shaft extending within the outer shaft and within an interior of the capsule. The transcatheter devices further comprise a distal tip attached to a distal end of the inner shaft, wherein the distal tip is configured to close a distal end of the capsule. The transcatheter device further comprises a leaflet sheath slidably disposed over the capsule and at least one leaflet member. The leaflet sheath is configured to be retracted relative to the capsule to radially expand distal portions of the leaflet member relative to the inner shaft to be positioned outside of the leaflet sheath. The leaflet sheath is further configured to be extended relative to the capsule to radially retract the distal portions of the leaflet member relative to the inner shaft to be positioned within the leaflet sheath.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates a side view of a stented prosthesis in an expanded orientation in accordance with aspects of the disclosure;

FIG. 2 schematically illustrates the stented prosthesis of FIG. 1 in a contracted orientation;

FIG. 3 illustrates a schematic exploded view of a transcatheter device in accordance with aspects of the disclosure;

FIG. 4 illustrates a partial top perspective view of a distal end portion of a transcatheter device in accordance with embodiments of the disclosure;

FIG. 5 is a top view of the distal end portion of the transcatheter device of FIGS. 4 and 11;

FIGS. 6-10 schematically illustrate example methods of deploying a stented prosthesis within a heart valve with the transcatheter device of FIGS. 4-5;

FIGS. 11-13 schematically illustrate example methods of deploying a stented prosthesis within a heart valve with a transcatheter device in accordance with further aspects of the disclosure;

FIGS. 14-17 illustrate alternative embodiments of leaflet members radially expanding from a distal end of a leaflet sheath in accordance with aspects of the disclosure; and

FIGS. 18-21 schematically illustrate example methods of deploying a stented prosthesis with a transcatheter device comprising leaflet members in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

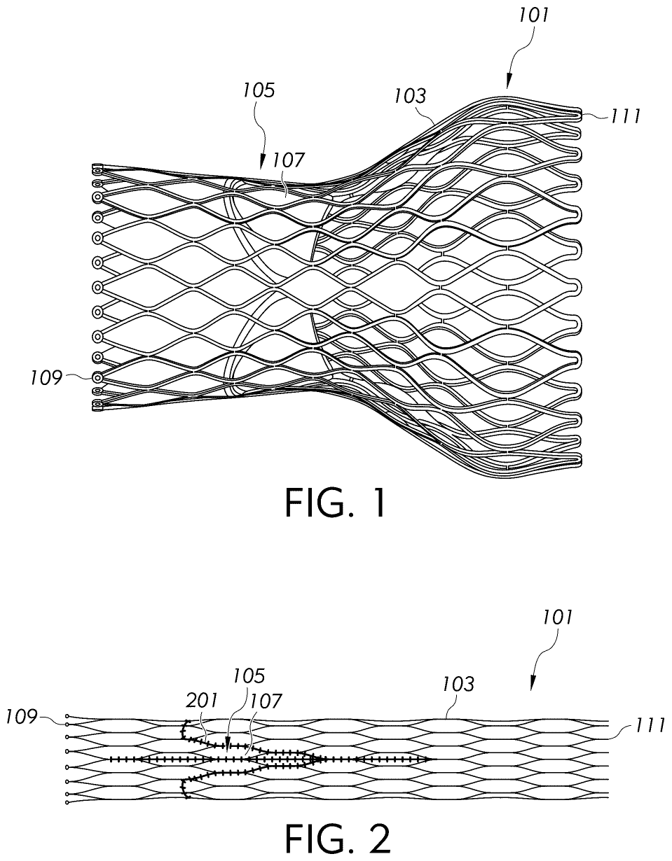

FIG. 1 illustrates a side view of a stented prosthesis 101 in an expanded orientation. The stented prosthesis 101 includes expandable stent frame 103 and a valve structure 105 that comprises a plurality of leaflets 107 (e.g., two or three leaflets). The expandable stent frame 103 of the stented prosthesis 101 supports the valve structure 105 within a lumen defined by the expandable stent frame 103. As shown in FIG. 2, a plurality of sutures 201 can mount the valve structure 105 to the expandable stent frame 103 wherein the valve structure 105 can be collapsed within the expandable stent frame 103 in the collapsed orientation shown in FIG. 2. Furthermore, as shown in FIG. 1, the valve structure can be deployed to operate as a valve (e.g., heart valve) when the stented prosthesis 101 radially expanded to the expanded orientation. In the example stented prosthesis 101 is balloon-expandable or self-expanding and may be implanted at a treatment site within a patient to replace a preexisting aortic valve, a pulmonic valve, a mitral valve, or a tricuspid valve. The preexisting valve to be replaced may be a native valve or a previously implanted prosthetic valve, such as a failed surgical replacement valve or a failed preexisting stented prosthesis.

As shown in FIGS. 1-2, the stented prosthesis 101 includes an inflow end 109 and an outflow end 111. The leaflets 107 are attached to the expandable stent frame 103 such that when pressure at the inflow end 109 exceeds pressure at the outflow end 111, the leaflets 107 open to allow blood flow through the stented prosthesis 101 from the inflow end 109 to the outflow end 111. When the pressure at the outflow end 111 exceeds pressure at the inflow end 109, the leaflets 107 close to prevent blood flow from the outflow end 111 to the inflow end 109.

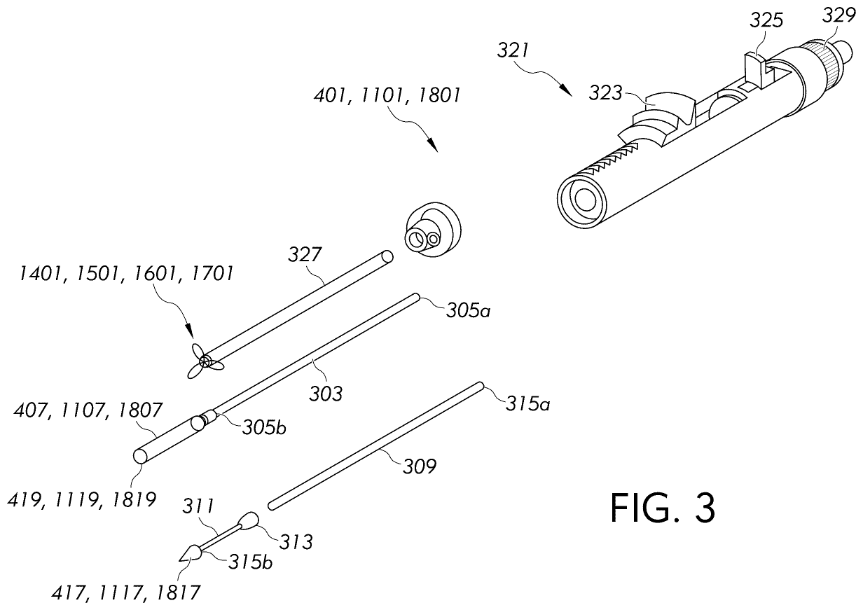

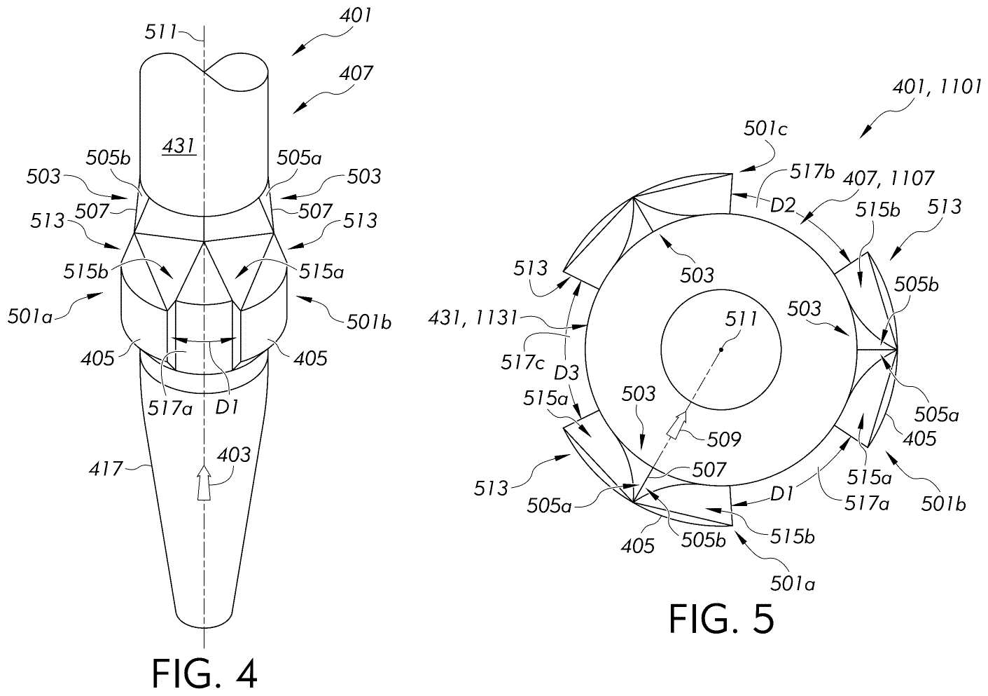

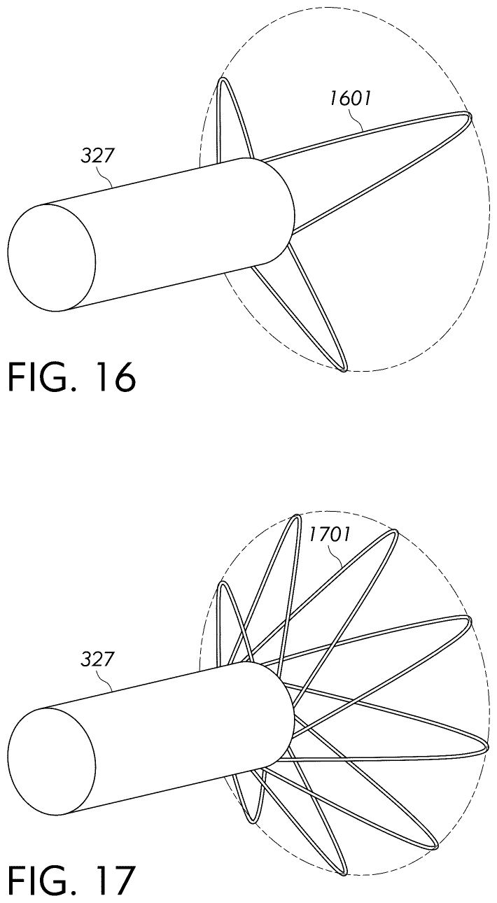

FIG. 3 is an exploded schematic view of various transcatheter devices 401, 1101, 1801 (e.g., transcatheter delivery systems) in accordance with aspects of the disclosure illustrated and discussed more fully with respect to FIGS. 4-21. For example, the transcatheter devices 401, 1101, 1801 of FIGS. 4-21 can each comprise an outer shaft 303 comprising a proximal end 305a and a distal end 305b attached to a capsule 407, 1107, 1807. The transcatheter devices 401, 1101, 1801 can each further comprise an inner shaft 309 that can extend within the outer shaft 303. The inner shaft 309 can comprise a distal segment 311 with a retainer device 313 configured to releasably engage a stented prosthesis in the collapsed orientation within the capsule 407, 1107, 1807 while it is mounted on the inner shaft 309. The inner shaft 309 can comprise a proximal end 315a and a distal end 315b. The distal end 315b of the inner shaft 309 can be attached to a distal tip 417, 1117, 1817 of the transcatheter device 401, 1101, 1801. The distal tip 417, 1117, 1817 is configured to close a distal end 419, 1119, 1819 of the capsule 407, 1107, 1807 in the delivery position. In some embodiments, the transcatheter devices 401, 1101, 1801 can each further comprise a handle apparatus 321. The proximal end 305a of the outer shaft 303 and the proximal end 315a of the inner shaft 309 can both be secured to the handle apparatus 321 to facilitate manipulation of the transcatheter device 401, 1101, 1801 during a surgical procedure by a surgeon.

With further reference to the transcatheter devices 401, 1101 of FIGS. 4-13, the capsule 407, 1107 of each transcatheter device 401, 1101 can comprise an outer circumferential surface 431, 1131. The outer circumferential surface can comprise a wide range of cylinder shapes. As shown, the outer circumferential surfaces 431, 1131 can comprise the same shaped cylindrical surface. For example, as shown in FIGS. 4-5, and 11, the outer circumferential surfaces 431, 1131 can comprise a circular cylindrical surface.

The transcatheter devices 401, 1101 can further comprise at least one protrusion extending radially outwardly from the outer circumferential surface. For example, as shown in FIG. 5, a plurality of protrusions 501a, 501b, 501c can be provided although one, two, or more than three protrusions can be provided in further embodiments. Still further, as shown, the plurality of protrusions 501a, 501b, 501c can be identical protrusions although the protrusions may have different configurations (e.g., shapes, sizes) from one another in further embodiments). In some embodiments, the number of protrusions can match the number of leaflets the protrusions are designed to splay. For example, when splaying three leaflets of a tricuspid valve, there may be three protrusions 501a, 501b, 501c. Alternatively, there may be a different number of protrusions than the number of leaflets. For example, there may be two protrusions designed to splay the leaflets that may pose a blockage concern for the two coronary ostium located in the vicinity of the leaflets to be splayed.

In some embodiments, at least one protrusion can comprise at least one wedge. For example, as shown, each protrusion of the plurality of protrusions 501a, 501b, 501c can comprise a first wedge 503 comprising a pair of tapered surfaces 505a, 505b. The pair of tapered surfaces 505a, 505b taper in a proximal direction 403 (see FIG. 4) to an edge 507. As shown in FIG. 5, the edge extends in a direction comprising a directional component 509 that is substantially perpendicular to a central axis 511 of the capsule 407, 1107.

In some embodiments, in addition to the first wedge 503, each protrusion of the plurality of protrusions 501a, 501b, 501c can comprise a second wedge 513 comprising a pair of surfaces 515a, 515b that taper in the proximal direction 403 toward the first wedge 503. For instance, as shown in FIGS. 4-5, the pair of surface 515a, 515b of the second wedge 513 can taper such that portions meet at the distal end of the first wedge 503.

As shown in FIG. 5, each protrusion of the plurality of protrusions 501a, 501b, 501c are spaced apart from each of a corresponding pair of adjacent protrusions of the plurality of protrusions by a circumferential spaced distance. For example, a first protrusion 501a is spaced apart from each adjacent protrusion 501b, 501c of a corresponding pair of adjacent protrusions 501b, 501c by a circumferential spaced distance D1, D3; a second protrusion 501b is spaced apart from each adjacent protrusion 501c, 501a of a corresponding pair of adjacent protrusions 501c, 501a by a circumferential spaced distance D2, D1; and a third protrusion 501c is spaced apart from each adjacent protrusion 501a, 501b of a corresponding pair of adjacent protrusions 501a, 501b by a circumferential spaced distance D3, D2. As shown, in some embodiments, the spaced distances D1, D2, D3 can be substantially equal such that the plurality of protrusions are equally circumferentially spaced about the central axis 511 of the capsule 407, 1107 although different distances may be provided in further embodiments. As shown, the spaced apart plurality of protrusions 501a, 501b, 501c can defined a plurality of channels 517a, 517b, 517c defined between corresponding distal portions 405 of the plurality of protrusions 501a, 501b, 501c. As shown in FIG. 4, the plurality of channels 517a, 517b, 517c can each comprise a width defined between the corresponding adjacent distal portions 405 of the plurality of protrusions 501a, 501b, 501c equal to the corresponding circumferential spaced distance D1, D2, D3 with a length extending in the proximal direction 403 (e.g., proximal direction of the central axis 511) from the distal end 419, 1119 of the capsule 407, 1107 to the distal end of the corresponding second wedge 513. Thus, the at least one protrusion, such as the plurality of protrusions 501a, 501b, 501c can extend radially outwardly from a distal portion of the outer circumferential surface 431, 1131. As such, the splaying can result in movement of the portions of the leaflets into proper position just prior to pinning the moved leaflets in place with the deployed stented prosthesis as discussed more fully below.

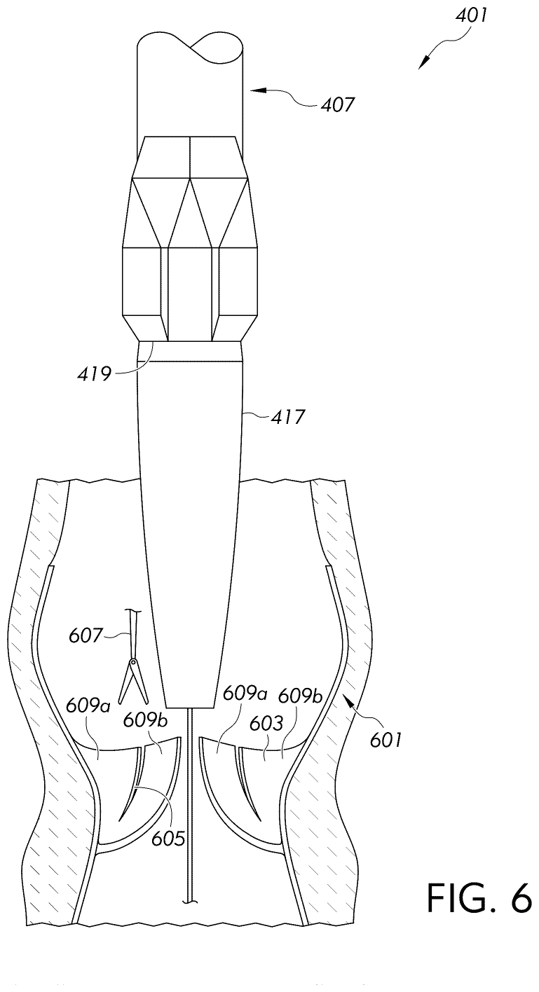

Methods of deploying the stented prosthesis 101 within a preexisting heart valve 601 will now be discussed with initial reference to the transcatheter device 401 shown in FIGS. 6-10. The preexisting valve 601 throughout all of the embodiments of the disclosure comprises a preexisting stented prosthesis that was previously implanted and is being functionally replaced with the stented prosthesis 101. In further embodiments, the preexisting heart valve 601 can comprise another medically previously modified or replaced valve. In still further embodiments, the preexisting heart valve 601 throughout all of the embodiments of the disclosure can comprise a native heart valve.

As shown schematically in FIG. 6, the method can begin by lacerating at least one leaflet 603, such as all of the leaflets 603 along laceration 605 with a laceration device 607. Once lacerated, each leaflet can be divided, for example, into two or more portions such as the illustrated first portion 609a and second portion 609b. As shown, the laceration can extend from the outer edge of the leaflet to the root of the leaflet. The method further includes delivering the stented prosthesis 101 while the stented prosthesis is mounted on the inner shaft 309 in the contracted orientation within an interior of the capsule 407 and while the distal end 419 of the capsule 407 is closed (see FIG. 6) by the distal tip 417.

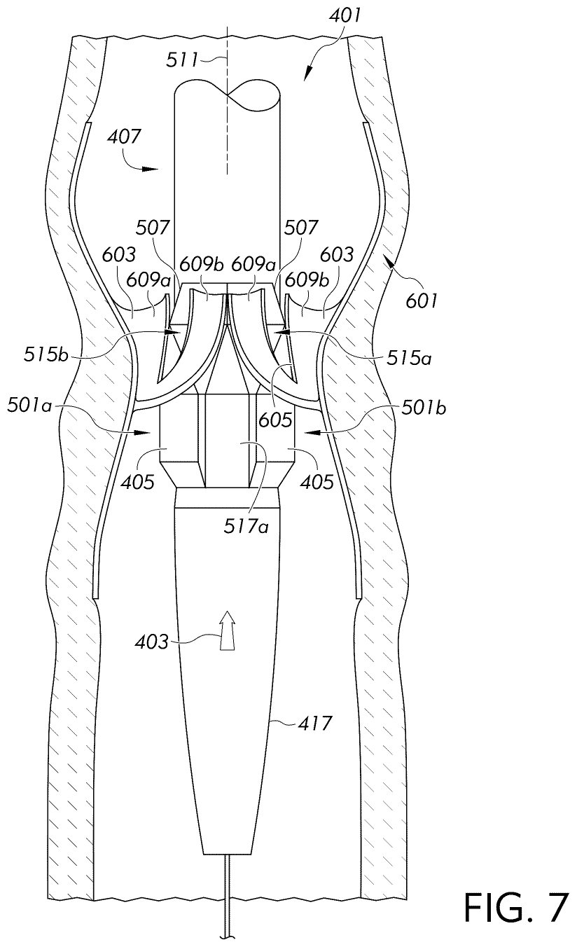

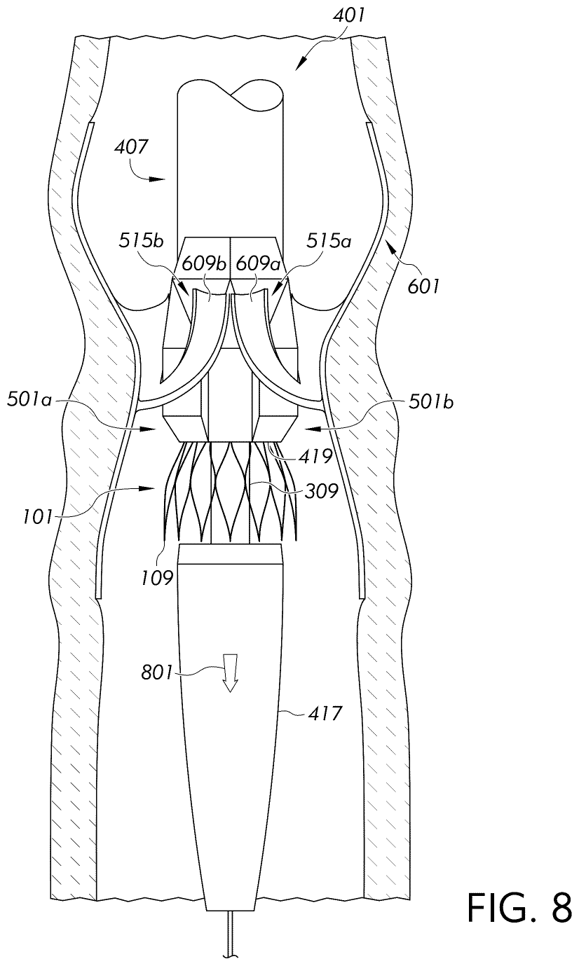

As shown in FIG. 7, the method can then include moving the at least one protrusion 501a, 501b, 501c relative to leaflets 603 of the heart valve 601 to splay the first portion 609a from the second portion 609b of the lacerated leaflet 603. For example, the protrusions 501a, 501b, 501c can be inserted past the preexisting valve structure of the preexisting heart valve 601 and retracted back in the proximal direction 403 of the central axis 511 of the capsule 407. In some embodiments, the proximal ends of the edges 507 of the first wedges 503 of the protrusions 501a, 501b, 501c can be aligned with the lacerations 605. As such, the edges 507 of the first wedges 503 can act the provide an initial gradual splaying of the first and second portions 609a, 609b of the leaflets 603 as the protrusions 501a, 501b, 501c are moved in the proximal direction 403.

As shown in FIG. 8, further movement of the protrusions 501a, 501b, 501c in the proximal direction 403 results in further splaying of the first and second portions 609a, 609b as the portions of the leaflets 603 ride on the surfaces 515a, 515b of the second wedges 513. As further shown in FIG. 8, the distal tip 417 can be moved in a distal direction 801 of the central axis 511 away from the capsule 407 to open the distal end 419 of the capsule 407. In some embodiments, as schematically shown in FIG. 3, an actuator 323 can be moved to cause the inner shaft 309 (together with the stented prosthesis 101 to distally move relative to the outer shaft 303 (and the capsule 407 attached to the distal end 305b of the outer shaft 303). Such movement of the actuator 323 can thereby distally move the distal tip 417 together with the stented prosthesis 101 in the distal direction 801 relative to the capsule 407, wherein the inflow end 109 and associated inflow portions of the stented prosthesis 101 begin to self expand outside of the distal end 419 of the capsule 407.

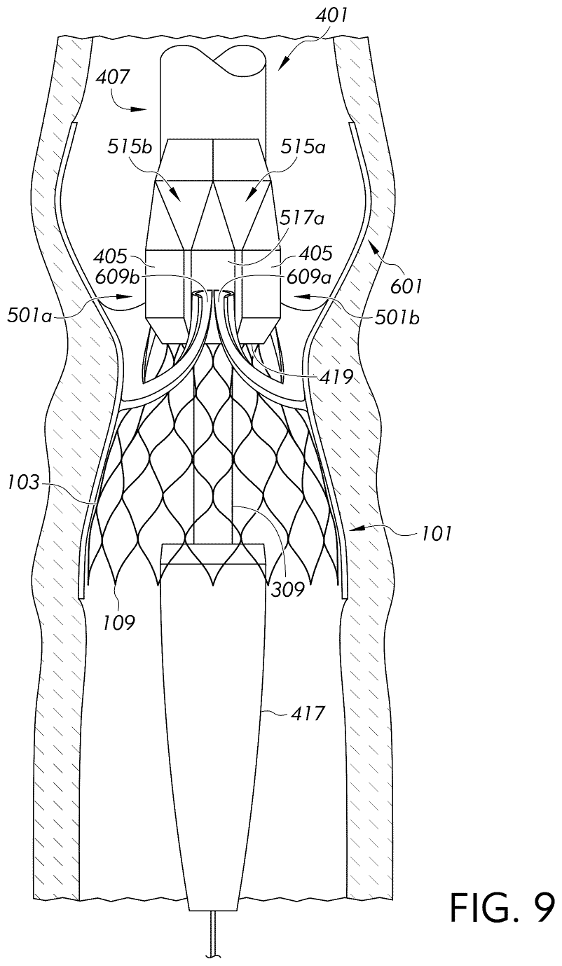

As shown in FIG. 9, further movement of the protrusions 501a, 501b, 501c in the proximal direction 403 results in portions 609a, 609b of adjacent leaflets 603 being dropped from the surfaces 515a, 515b of the second wedge 513 into a corresponding channel 517a, 517b, 517c between adjacent distal portions 405 of the protrusions 501a, 501b, 501c. Placing the portions 609a, 609b of adjacent leaflets 603 within the corresponding channels can help fully splay the leaflets and help hold the splayed orientation until the self-expanding stented prosthesis 101 is fully deployed. As further shown in FIG. 9, the distal tip 417 can be further moved distally away from the capsule 407 to further deploy the stented prosthesis 101 from the capsule 407. As shown, the inflow end 109 and inflow portions of the expandable stent frame 103 begin implanting with corresponding structures of the inflow portions of the preexisting heart valve 601.

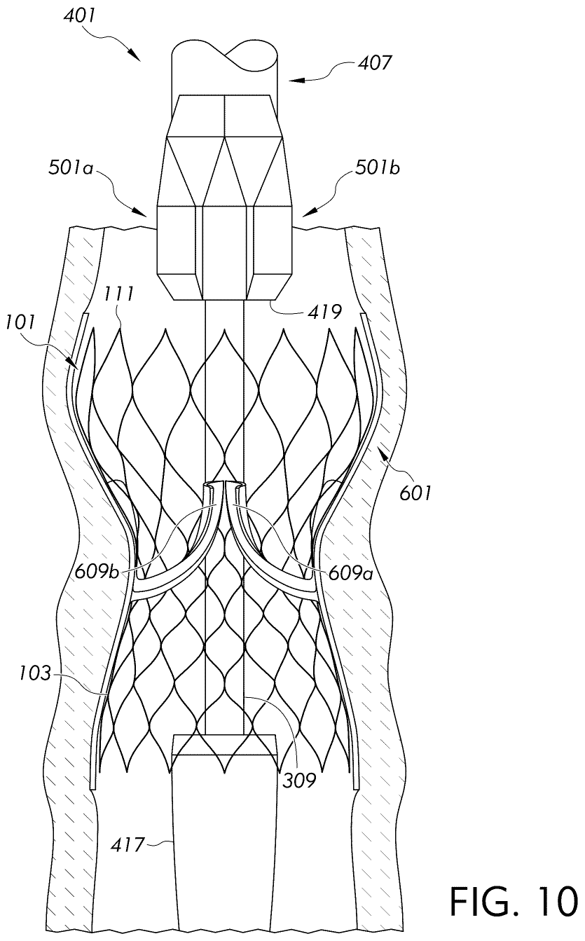

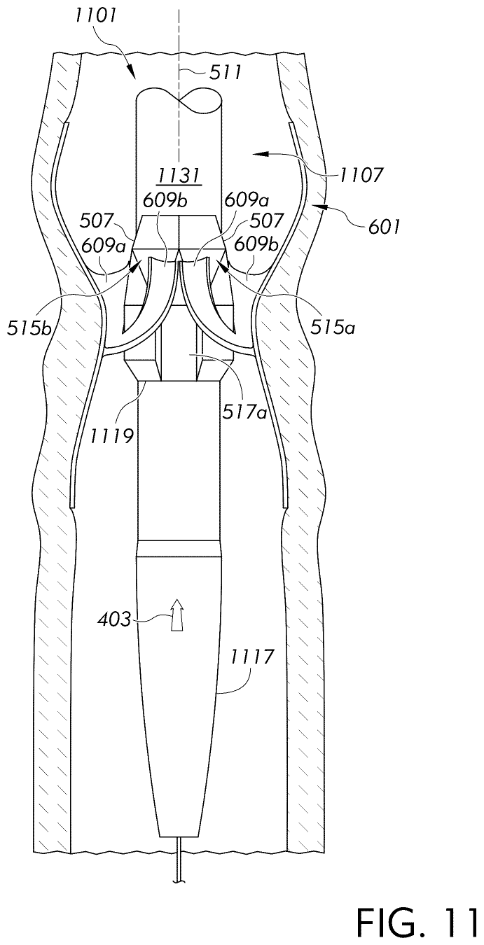

FIG. 10 illustrates the stented prosthesis 101 being fully deployed from the interior of the capsule 407 and implanted within the preexisting heart valve 601. As shown, the distal tip 417 has been moved further distally away from the capsule 407 wherein the outflow end and outflow end portions have fully exited the interior of the capsule 407. Furthermore, the first and second portions 609a, 609b of the splayed leaflets 603 have dropped out of the corresponding channels 517a, 517b, 517c and have thereafter been pinned (e.g., substantially immediately pinned) in position by the implanted stent prosthesis 101. Indeed, once the outflow end 111 of the expandable stent frame 103 leaves the interior of the capsule 407, the stent frame 103 quickly self expands to pin first and second portions 609a, 609b of the splayed leaflets 603 in position between the stented prosthesis 101 and the preexisting heart valve 601.

In some embodiments, the actuator 323 can be configured to alternatively move the outer shaft 303 proximally relative to stented prosthesis 101 and the inner shaft 309. Such movement of the actuator 323 can thereby proximally move the outer shaft 303 and capsule 407 while the inner shaft 309 and stented prosthesis 101 remain stationary, therefore exposing the inflow end 109 such that the inflow end 109 begins to expand while the splayed leaflets 603 ride on the surfaces of the wedges.

FIGS. 11-13 illustrate methods of deploying the stented prosthesis 101 within the heart valve 601 comprising the plurality of leaflets 603 with the transcatheter device 1101 that can be similar or identical to the method disclosed and described with respect to the transcatheter device 401 in FIGS. 6-9 unless otherwise stated. As mentioned previously, the method can begin by lacerating at least one leaflet 603, such as all of the leaflets 603 along laceration 605 with a laceration device 607 as discussed with respect to FIG. 6 above. Once lacerated, the method further includes delivering the stented prosthesis 101 while the stented prosthesis is mounted on the inner shaft 309 in the contracted orientation within an interior of the capsule 1107 and while the distal end 1119 of the capsule 1107 is closed (see FIG. 11) by the distal tip 1117.

The method can then include moving the at least one protrusion 501a, 501b, 501c relative to leaflets 603 of the heart valve 601 to splay the first portion 609a from the second portion 609b of the lacerated leaflet 603. For example, the protrusions 501a, 501b, 501c can be inserted past the preexisting valve structure of the preexisting heart valve 601 and retracted back in the proximal direction 403 of the central axis 511 of the capsule 1107. In some embodiments, the proximal ends of the edges 507 of the first wedges 503 of the protrusions 501a, 501b, 501c can be aligned with the lacerations 605. As such, the edges 507 of the first wedges 503 can act to provide an initial gradual splaying of the first and second portions 609a, 609b of the leaflets 603 as the protrusions 501a, 501b, 501c are moved in the proximal direction 403.

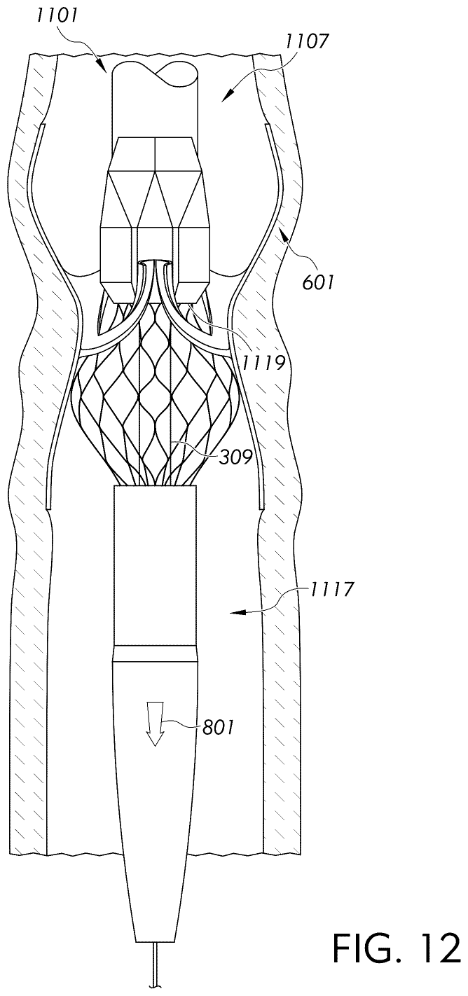

As shown in FIG. 11, further movement of the protrusions 501a, 501b, 501c in the proximal direction 403 results in further splaying of the first and second portions 609a, 609b as the portions of the leaflets 603 ride on the surfaces 515a, 515b of the second wedges 513. As further shown in FIG. 12, the distal tip 1117 can be moved in a distal direction 801 of the central axis 511 away from the capsule 1107 to open the distal end 1119 of the capsule 1107. In some embodiments, as schematically shown in FIG. 3, an actuator 323 can be moved to cause the inner shaft 309 (together with the stented prosthesis 101) to distally move relative to the outer shaft 303. Such movement of the actuator 323 can thereby distally move the distal tip 417 causing portions of the stented prosthesis 101 to be pulled from an interior of the capsule 1107. An intermediate portion between the capsule 1107 and the distal tip 1117 self expands while the inflow end 109 is retained in a compressed orientation within an interior area of the distal tip 1117 while an outflow end 111 of the stented prosthesis 101 is retained in a compressed orientation within an interior area of the capsule 1107. In further embodiments, the actuator 323 may alternatively be configured to cause the outer shaft 303 to move proximally relative to the stented prosthesis 101 while the inner shaft 309 and the stented prosthesis 101 remain stationary. Such movement can result in the splayed leaflets 603 riding on the surfaces 515a, 515b as the outer shaft 303 is moved proximally by the actuator 323 with inflow end 109 beginning to expand outside of the capsule.

Further movement of the protrusions 501a, 501b, 501c in the proximal direction 403 results in portions 609a, 609b of adjacent leaflets 603 being dropped from the surfaces 515a, 515b of the second wedge 513 into a corresponding channel 517a, 517b, 517c between adjacent distal portions 405 of the protrusions 501a, 501b, 501c. Placing the portions 609a, 609b of adjacent leaflets 603 within the corresponding channels can help fully splay the leaflets and help hold the splayed orientation until the self-expanding stented prosthesis 101 is fully deployed. The distal tip 1117 can be further moved distally away from the capsule 407 to further deploy the stented prosthesis 101 from the capsule 407.

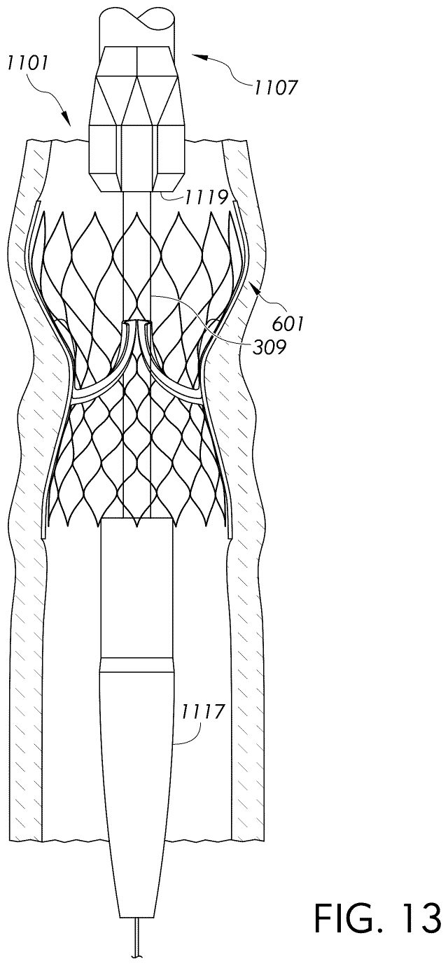

FIG. 13 illustrates the stented prosthesis 101 being fully deployed from the interior of the capsule 1107 and implanted within the preexisting heart valve 601. As shown, the distal tip 1117 has been moved further distally away from the capsule 1107 wherein the outflow end and outflow end portions have fully exited the interior of the capsule 1107. Furthermore, the first and second portions 609a, 609b of the splayed leaflets 603 have dropped out of the corresponding channels 517a, 517b, 517c and have thereafter been pinned (e.g., substantially immediately pinned) in position by the implanted stent prosthesis 101. Indeed, once the outflow end 111 of the expandable stent frame 103 leaves the interior of the capsule 1107, the stent frame 103 quickly self expands to pin first and second portions 609a, 609b of the splayed leaflets 603 in position between the stented prosthesis 101 and the preexisting heart valve 601.

In some embodiments, moving the at least one protrusion 501a, 501b, 501c relative to the leaflets 603 can be provided by proximally retracting the capsule 1107 relative to the stented prosthesis 101. For example, referring to FIG. 3, the handle apparatus 321 can comprise a second actuator 325 configured to proximally retract the capsule 1107 relative to the stented prosthesis 101. In some embodiments, such proximal retracting of the capsule can also result in opening of the distal end of the capsule. In still further embodiments, the distal tip can be distally extended while the capsule is proximally retracted to deploy the self-expanding stent frame.

As shown in FIG. 3, in some embodiments, the transcatheter device 1801 can comprise the outer shaft 303 and the capsule 1807 attached to the distal end 305b of the outer shaft 303. The transcatheter device 1801 further comprises the inner shaft 309 extending within the outer shaft 303 and within an interior of the capsule 1807. The transcatheter device 1801 further comprises the distal tip 1817 attached to the distal end 315b of the inner shaft 309, wherein the distal tip 1817 is configured to close the distal end 1819 of the capsule 1807. The transcatheter device 1801 further comprises a leaflet sheath 327 slidably disposed over the capsule 1807 wherein the outer shaft 303 extends within the leaflet sheath 327. The transcatheter device 1801 further comprises at least one leaflet member 1401, 1501, 1601, 1701. The leaflet sheath 327 is configured to be retracted relative to the capsule 1807 to radially expand the distal portions of the at least one leaflet member 1401, 1501, 1601, 1701. The leaflet sheath 327 is configured to be retracted relative to the capsule 1807 to radially expand distal portions of the leaflet member 1401, 1501, 1601, 1701 relative to the inner shaft 309 to be positioned outside of the leaflet sheath 327. The leaflet sheath 327 is configured to be extended relative to the capsule 1807 to radially retract the distal portions of the leaflet member 1401, 1501, 1601, 1701 relative to the inner shaft 309 to be positioned within the leaflet sheath 327.

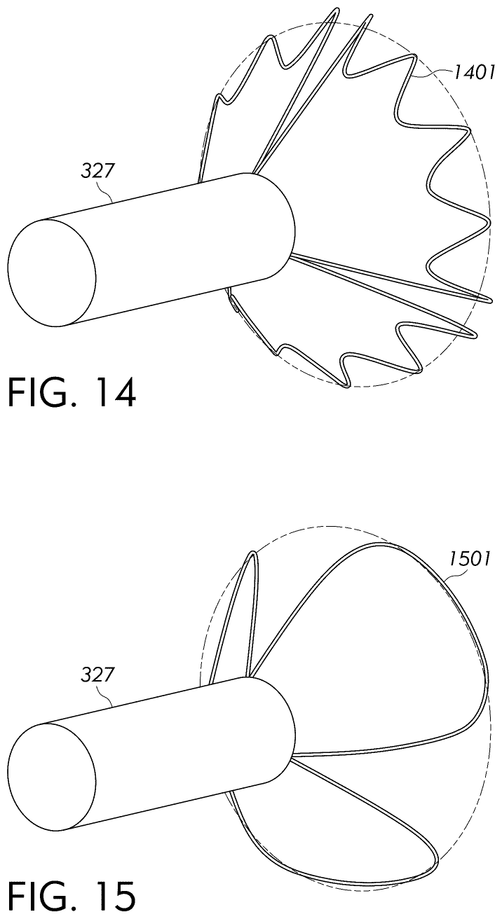

As shown, the leaflet members 1401, 1501, 1601, 1701 can comprise a wire. The wire can comprise a plurality of separate wires bundled together or can comprise a plurality of wire segments that are attached to one another. In further embodiments, as shown, the wire can comprise at least one continuous wire segment that is bent into one or a plurality of prongs. As shown in FIGS. 14-16, the leaflet members 1401, 1501, 1601 can comprise three circumferentially arranged prongs. In some embodiments, each prong can comprise a loop although a single protrusion may be provided in further embodiments. Providing the prongs as loops can help strengthen the rigidity of the prong and can also help generate a plurality of prongs (e.g., loops) with a single continuous length of wire to simplify and reduce production costs. Three prongs (e.g., loops) are illustrated in FIGS. 14-16 although a single prong, two prongs, or more than three prongs may be provided in further embodiments. As shown in FIG. 17, for example, the leaflet members 1701 can comprise a plurality of wire prongs that are greater than three prongs (e.g., greater than three wire loops). Furthermore, as shown in FIG. 17, the base of the wire loops of adjacent wire loops can overlap each other to provide enhanced rigidity to the overall leaflet member arrangement. In the illustrated embodiment, each loop can comprise a single wire. In further embodiments, all of the wire prongs can comprise a single wire bent into leaflet member arrangement. As shown in FIGS. 14-17, each prong (e.g., loop) of the leaflet members 1401, 1501, 1601, 1701 can be identical to the remaining prongs (e.g., loops) of the plurality of prongs (e.g., loops). Furthermore, if the prongs comprise loops, the outermost flared portion of the loops can comprise a single bend as shown by the leaflet members 1501, 1601, 1701 of FIGS. 15-17. Alternatively, the outermost flared portion of the loops can comprise a sinusoidal distal portion as shown by the leaflet member 1401 of FIG. 14. The sinusoidal distal portion can help each prong act as a grappling member to grip portions of the leaflets in use.

The leaflet members can comprise a self-expanding material comprises such as nitinol although other self-expanding material may be provided in further embodiments. The leaflet members 1401, 1501, 1601, 1701 can comprise a normal, relaxed state that is radially expanded as shown in FIGS. 14-16. The leaflet members 1401, 1501, 1601, 1701 can be radially compressed within the leaflet sheath 327. Once the leaflet sheath 327 is retracted, the leaflet members 1401, 1501, 1601, 1701 can self expand to its normal, relaxed state illustrated in FIGS. 14-16.

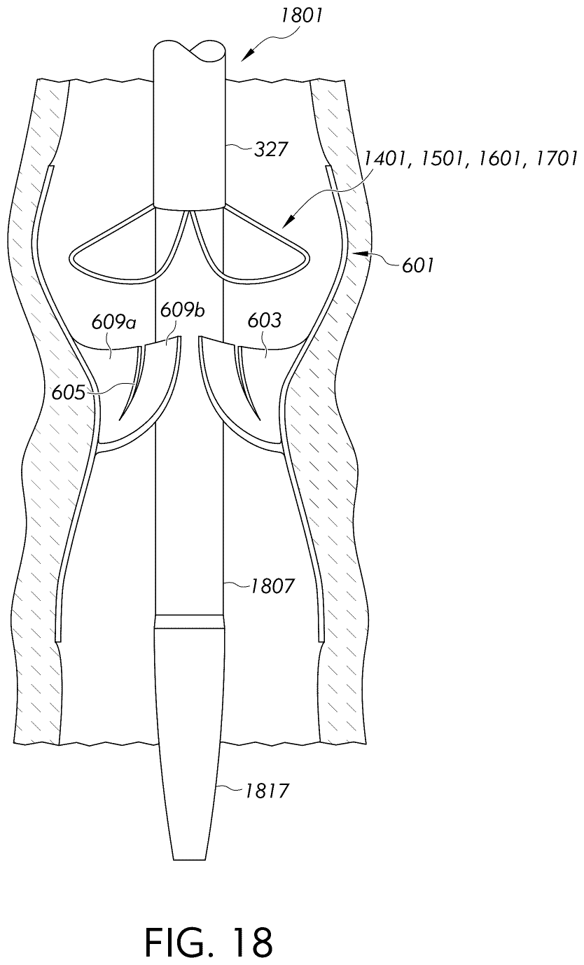

Methods of deploying the stented prosthesis 101 within the heart valve 601 with the transcatheter device 1801 will now be described with reference to FIGS. 18-21. As with the previous embodiments, the heart valve 601 is illustrated as a preexisting stented prosthesis although the heart valve can comprise a native heart valve in further embodiments. As mentioned previously, the method can begin by lacerating at least one leaflet 603, such as all of the leaflets 603 along laceration 605 with a laceration device 607 as discussed with respect to FIG. 6 above. Once lacerated, the method further includes delivering the stented prosthesis 101 to the heart valve 601 while the stented prosthesis 101 is mounted on the inner shaft 309 within the interior of the capsule 1807 and while the distal end 1819 of the capsule 1807 is closed by the distal tip 1817.

As shown, in FIG. 18, the leaflet sheath 327 can be retracted relative to the capsule 1807 to radially expand the distal portions of the leaflet member 1401, 1501, 1601, 1701 relative to the inner shaft 309 to be positioned outside of the leaflet sheath 327. In some embodiments, as schematically shown in FIG. 3, a third actuator 329 can be engaged by a surgeon to retract the leaflet sheath 327 relative to the capsule 1807.

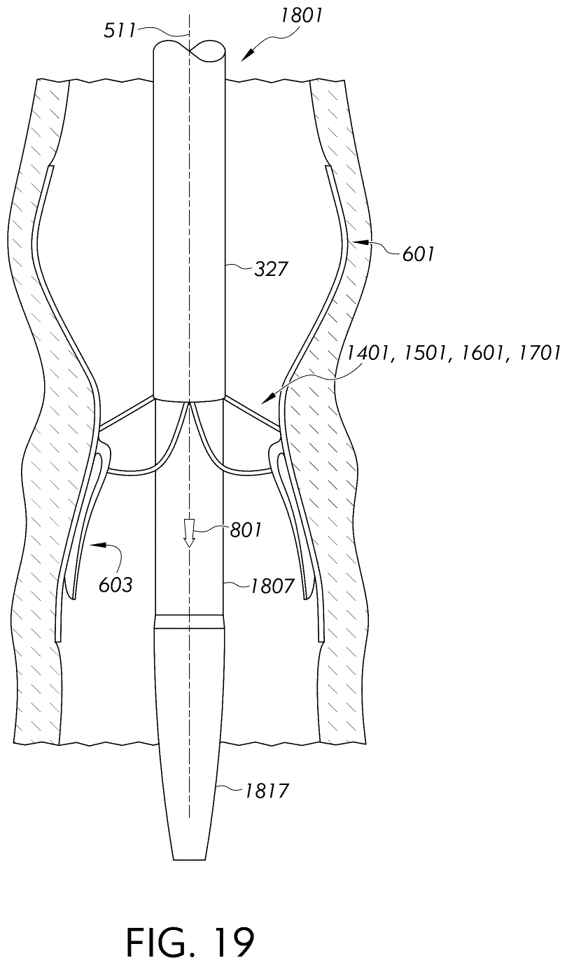

As shown in FIG. 19, the leaflet sheath 327 may be extended in the distal direction 801 relative to the capsule 1807 (e.g., while the capsule 1807 remains relatively stationary relative to the leaflets 603) such that the leaflet members 1401, 1501, 1601, 1701 are moved to displace portions of the plurality of leaflets 603 of the heart valve 601. Indeed, as shown, the leaflet members 1401, 1501, 1601, 1701 can be moved to press the leaflets downward in the distal direction 801 of the central axis 511. For example, the surgeon may again engage the third actuator 329 to cause shifting of the leaflet sheath 327 (together with the leaflet members 1401, 1501, 1601, 1701) in the distal direction 801 of the central axis 511 relative to the capsule 1807.

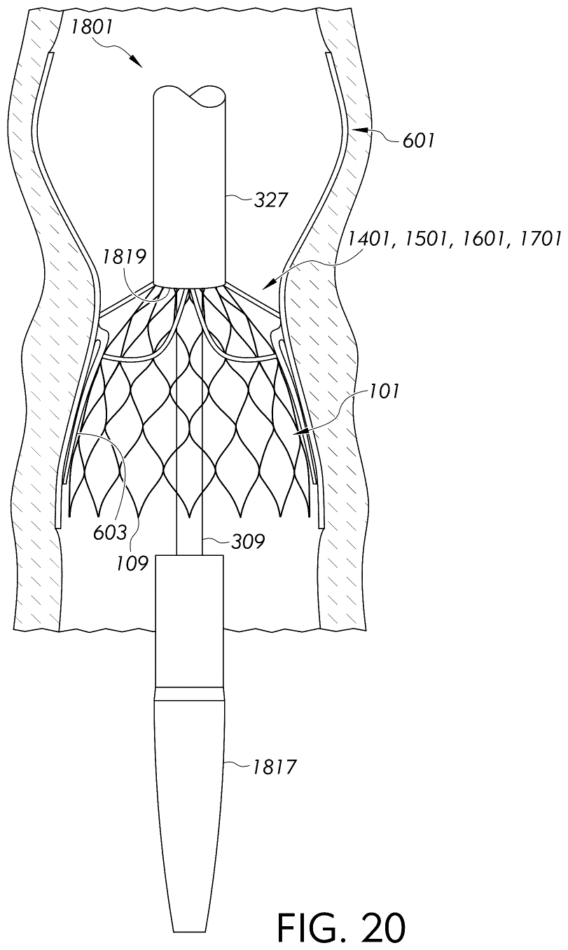

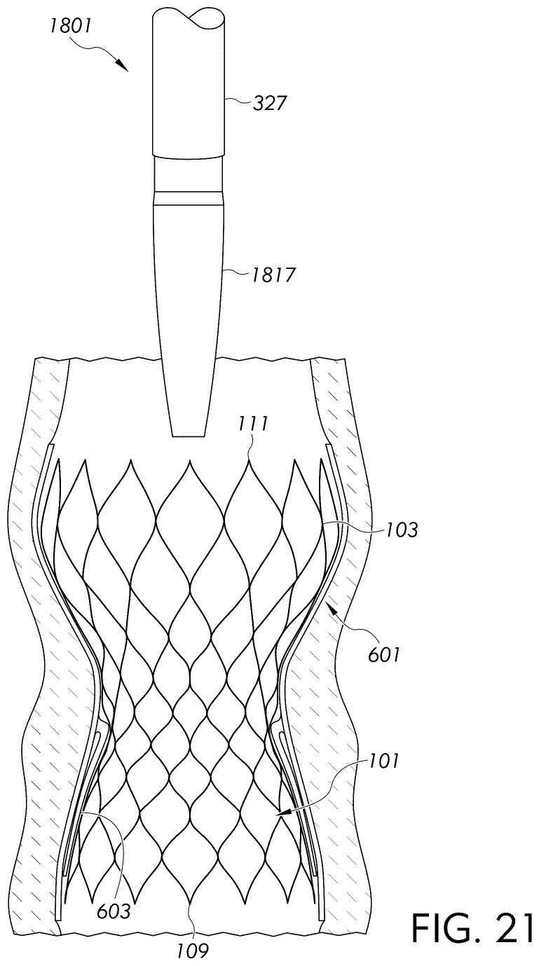

As shown in FIGS. 20-21, the stented prosthesis 101 may be deployed from the interior of the capsule 1807, wherein the displaced leaflets 603 are pinned in position by the deployed stented prosthesis 101. For example, a surgeon may engage the first actuator 323 of the handle apparatus 321 to retract the capsule 1807 from the distal tip 1817 to deploy the stented prosthesis 101. As shown in FIG. 21, once the stented prosthesis 101 is fully implanted in the preexisting heart valve 601, the distal tip 1817 may be retracted to close the distal end 1819 of the capsule 1807 while the leaflet sheath 327 slides back over the capsule 1807 to collapse the leaflet member 1401, 1501, 1601, 1701 to prevent interference when removing the transcatheter device 1801 from the vasculature of the patient.

In accordance with the disclosure, non-limiting aspects of the disclosure will now be described. Various combinations of the aspects can be provided in accordance with the disclosure.

Aspect 1. A transcatheter device comprises an outer shaft and a capsule attached to a distal end of the outer shaft. The capsule comprises an outer circumferential surface and at least one protrusion extending radially outwardly from the outer circumferential surface. The transcatheter device further comprises an inner shaft extending within the outer shaft, and a distal tip attached to a distal end of the inner shaft, wherein the distal tip is configured to close a distal end of the capsule.

Aspect 2. The transcatheter device of aspect 1, wherein the outer circumferential surface comprises a cylindrical surface.

Aspect 3. The transcatheter device of any one of aspects 1-2, wherein the outer circumferential surface comprises a circular cylindrical surface.

Aspect 4. The transcatheter device of any one of aspects 1-3, wherein the at least one protrusion extends radially outwardly from a distal portion of the outer circumferential surface.

Aspect 5. The transcatheter device of any one of aspects 1-4, wherein the at least one protrusion comprises a plurality of protrusions.

Aspect 6. The transcatheter device of any one of aspects 1-5, wherein each protrusion of the plurality of protrusions are spaced apart from each of a corresponding pair of adjacent protrusions of the plurality of protrusions by a circumferential spaced distance.

Aspect 7. The transcatheter device of any one of aspects 1-6, wherein the circumferentially spaced distances of the plurality of spaced apart protrusions are substantially equal.

Aspect 8. The transcatheter device of any one of aspects 1-7, further comprising a channel positioned between each adjacent pair of protrusions of the plurality of protrusions.

Aspect 9. The transcatheter device of any one of aspects 1-8, wherein the at least one protrusion comprises at least one wedge.

Aspect 10. The transcatheter device of any one of aspects 1-9, wherein the wedge comprises a pair of surfaces that taper to an edge.

Aspect 11. The transcatheter device of any one of aspects 1-10, wherein the pair of surfaces taper in a proximal direction to the edge.

Aspect 12. The transcatheter device of any one of aspects 1-11, wherein the edge extends in a direction comprising a directional component that is substantially perpendicular to a central axis of the capsule.

Aspect 13. Methods of deploying a stented prosthesis within a heart valve comprising a plurality of leaflets with a transcatheter device of aspect 1 comprise lacerating at least one leaflet of the plurality of leaflets into a first portion and a second portion. Methods comprise delivering the stented prosthesis to the heart valve while the stented prosthesis is mounted on the inner shaft within an interior of the capsule and while the distal end of the capsule is closed by the distal tip. Methods comprise moving the at least one protrusion relative to leaflets of the heart valve to splay the first portion from the second portion of the lacerated leaflets. Methods comprise deploying the stented prosthesis from the interior of the capsule, wherein the stented prosthesis is implanted within the heart valve and the first portion and second portion of the splayed leaflets are pinned in position by the implanted stented prosthesis.

Aspect 14. The method of aspect 13, wherein the heart valve comprises a preexisting prosthetic heart valve.

Aspect 15. The method of any one of aspects 13-14, wherein moving the at least one protrusion relative to the leaflets is provided by proximally retracting the capsule relative to the stented prosthesis.

Aspect 16. The method of aspect 15, wherein proximally retracting the capsule relative to the prosthesis opens the distal end of the capsule and deploys at least a portion of the stented prosthesis from the interior of the capsule.

Aspect 17. The method of any one of aspects 13-16, further comprising distally extending the distal tip relative to the capsule to deploy a distal portion of the stented prosthesis from an interior of the distal tip.

Aspect 18. A transcatheter device comprises an outer shaft and a capsule attached to a distal end of the outer shaft. The transcatheter device further comprises an inner shaft extending within the outer shaft and within an interior of the capsule. The transcatheter device further comprises a distal tip attached to a distal end of the inner shaft, wherein the distal tip is configured to close a distal end of the capsule. The transcatheter device further comprises a leaflet sheath slidably disposed over the capsule and at least one leaflet member. The leaflet sheath is configured to be retracted relative to the capsule to radially expand distal portions of the leaflet member relative to the inner shaft to be positioned outside of the leaflet sheath. The leaflet sheath is further configured to be extended relative to the capsule to radially retract the distal portions of the leaflet member relative to the inner shaft to be positioned within the leaflet sheath.

Aspect 19. The transcatheter device of aspect 18, wherein the at least one leaflet member comprises a self-expanding material.

Aspect 20. The transcatheter device of aspect 19, wherein the self-expanding material comprises nitinol.

Aspect 21. The transcatheter device of any one of aspects 18-20, wherein the leaflet member comprises a wire.

Aspect 22. The transcatheter device of aspect 21, wherein the wire is bent into a plurality of prongs.

Aspect 23. The transcatheter device of aspect 22, wherein each prong of the plurality of prongs is identical to the remaining prongs of the plurality of prongs.

Aspect 24. The transcatheter device of any one of aspects 22-23, wherein each prong of the plurality of prongs comprises a wire loop.

Aspect 25. The transcatheter device of aspect 24, wherein the wire loop comprises a sinusoidal distal portion.

Aspect 26. A method of deploying a stented prosthesis within a heart valve with the transcatheter device of aspect 18 comprises delivering the stented prosthesis to the heart valve while the stented prosthesis is mounted on the inner shaft within the interior of the capsule and while the distal end of the capsule is closed by the distal tip. The method further comprises retracting the leaflet sheath relative to the capsule to radially expand the distal portions of the leaflet member relative to the inner shaft to be positioned outside of the leaflet sheath. The method further comprises moving the leaflet member to displace portions of a plurality of leaflets of the heart valve, and deploying the stented prosthesis from the interior of the capsule, wherein the displaced leaflets are pinned in position by the deployed stented prosthesis.

Aspect 27. The method of aspect 26, wherein the heart valve comprises a native heart valve.

Aspect 28. The method of any one of aspects 26-27, further comprising lacerating at least one leaflet of the plurality of leaflets prior to moving the leaflet member to displace the portions of the plurality of leaflets of the heart valve.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A transcatheter device comprising:
an outer shaft;
a capsule attached to a distal end of the outer shaft, the capsule comprising an outer circumferential surface and at least one protrusion extending radially outwardly from the outer circumferential surface;
an inner shaft extending within the outer shaft; and
a distal tip attached to a distal end of the inner shaft, wherein the distal tip is configured to close a distal end of the capsule.

2. The transcatheter device of claim 1, wherein the at least one protrusion extends radially outwardly from a distal portion of the outer circumferential surface.

3. The transcatheter device of claim 1, wherein the at least one protrusion comprises a plurality of protrusions.

4. The transcatheter device of claim 3, wherein each protrusion of the plurality of protrusions are spaced apart from each of a corresponding pair of adjacent protrusions of the plurality of protrusions by a circumferential spaced distance.

5. The transcatheter device of claim 3, further comprising a channel positioned between each adjacent pair of protrusions of the plurality of protrusions.

6. The transcatheter device of claim 1, wherein the at least one protrusion comprises at least one wedge.

7. The transcatheter device of claim 6, wherein the wedge comprises a pair of surfaces that taper to an edge.

8. The transcatheter device of claim 7, wherein the pair of surfaces taper in a proximal direction to the edge.

9. A method of deploying a stented prosthesis within a heart valve comprising a plurality of leaflets with the transcatheter device of claim 1 comprising:
lacerating at least one leaflet of the plurality of leaflets into a first portion and a second portion;
delivering the stented prosthesis to the heart valve while the stented prosthesis is mounted on the inner shaft within an interior of the capsule and while the distal end of the capsule is closed by the distal tip;
moving the at least one protrusion relative to leaflets of the heart valve to splay the first portion from the second portion of the lacerated leaflet; and
deploying the stented prosthesis from the interior of the capsule, wherein the stented prosthesis is implanted within the heart valve and the first portion and second portion of the splayed leaflets are pinned in position by the implanted stented prosthesis.

10. The method of claim 9, wherein the heart valve comprises a preexisting prosthetic heart valve.

11. The method of claim 9, wherein moving the at least one protrusion relative to the leaflets is provided by proximally retracting the capsule relative to the stented prosthesis.

12. The method of claim 11, wherein proximally retracting the capsule relative to the prosthesis opens the distal end of the capsule and deploys at least a portion of the stented prosthesis from the interior of the capsule.

13. The method of claim 9, further comprising distally extending the distal tip relative to the capsule to deploy a distal portion of the stented prosthesis from an interior of the distal tip.

14. A transcatheter device comprising:
an outer shaft;
a capsule attached to a distal end of the outer shaft;
an inner shaft extending within the outer shaft and within an interior of the capsule;
a distal tip attached to a distal end of the inner shaft, wherein the distal tip is configured to close a distal end of the capsule;
a leaflet sheath slidably disposed over the capsule; and
at least one leaflet member, wherein the leaflet sheath is configured to be retracted relative to the capsule to radially expand distal portions of the leaflet member relative to the inner shaft to be positioned outside of the leaflet sheath, and wherein the leaflet sheath is configured to be extended relative to the capsule to radially retract the distal portions of the leaflet member relative to the inner shaft to be positioned within the leaflet sheath.

15. The transcatheter device of claim 14, wherein the at least one leaflet member comprises a self-expanding material.

16. The transcatheter device of claim 14, wherein the leaflet member comprises a wire.

17. The transcatheter device of claim 16, wherein the wire is bent into a plurality of prongs.

18. A method of deploying a stented prosthesis within a heart valve with the transcatheter device of claim 14 comprising:
delivering the stented prosthesis to the heart valve while the stented prosthesis is mounted on the inner shaft within the interior of the capsule and while the distal end of the capsule is closed by the distal tip;
retracting the leaflet sheath relative to the capsule to radially expand the distal portions of the leaflet member relative to the inner shaft to be positioned outside of the leaflet sheath;
moving the leaflet member to displace portions of a plurality of leaflets of the heart valve;
deploying the stented prosthesis from the interior of the capsule, wherein the displaced leaflets are pinned in position by the deployed stented prosthesis.

19. The method of claim 18, wherein the heart valve comprises a preexisting prosthetic heart valve.

20. The method of claim 18, further comprising lacerating at least one leaflet of the plurality of leaflets prior to moving the leaflet member to displace the portions of the plurality of leaflets of the heart valve.
